(19) United States
(12) Patent Application Publication
(10) Pub. No.: US 2026/0198868 A1
(43) Pub. Date: Jul. 16, 2026

(54) APPARATUS AND METHOD FOR PREDICTING INTRADIALYTIC HYPOTENSION

(71) Applicant: ELECTRONICS AND TELECOMMUNICATIONS RESEARCH INSTITUTE, Daejeon (KR)

(72) Inventors: Tae Wuk BAE, Daejeon (KR); Kee Koo KWON, Daegu (KR); Kwang Yong KIM, Daejeon (KR); Woo Jin BYUN, Daejeon (KR)

(73) Assignee: ELECTRONICS AND TELECOMMUNICATIONS RESEARCH INSTITUTE, Daejeon (KR)

(21) Appl. No.: 19/449,898

(22) Filed: Jan. 15, 2026

(30) Foreign Application Priority Data
Jan. 15, 2025 (KR) 10-2025-0006345

(51) Int. Cl. A61B 5/00; A61B 5/024; A61M 1/14; G16H 50/30

(52) U.S. Cl. CPC A61B 5/7275; A61B 5/02405; A61M 1/14; G16H 50/30

(57) ABSTRACT

The present disclosure relates to an intradialytic hypotension prediction apparatus, the apparatus comprising: a processor; and a memory storing instructions executed by the processor, wherein the processor is configured to extract a predetermined parameter from a pulse signal representing a change in a pulse of a hemodialysis patient, and predict intradialytic hypotension by applying the parameter to a pre-trained prediction model.

### TECHNICAL FIELD

The present disclosure relates to an apparatus and method for predicting intradialytic hypotension.

### BACKGROUND

Intradialytic hypotension (IDH), which is defined as a rapid decrease in blood pressure during dialysis, poses a significant risk to dialysis patients. Intradialytic hypotension complicates approximately 15% to 30% of all hemodialysis treatments and occurs in more than 50% of treatments in a smaller number of patients.

Intradialytic hypotension is reported to occur in approximatiely 15-20% of patients during dialysis treatment. Intradialytic hypotension is defined as a decrease of 10 mmHg or more in mean arterial pressure, or a decrease of 20 mmHg or more in systolic blood pressure. Intradialytic hypotension may pose various risks to the heart, central nervous system, renal system, and gastrointestinal system, and the frequent occurrence of intradialytic hypotension may be an important risk factor explaining the mortality rate of dialysis patients. Furthermore, intradialytic hypotension may cause arrhythmia, which may have a significant impact on sudden cardiac death. Therefore, predicting intradialytic hypotension is critically important for hemodialysis patients.

In studies on intradialytic hypotension, prediction has been performed by using static data such as a patient's basic information, underlying diseases, and in-vivo chemical compound components during dialysis.

The background art of the present disclosure is disclosed in Korean Patent No. 10-2541362 (registered on June. 2, 2023).

### SUMMARY

An object of the present disclosure is to provide an intradialytic hypotension prediction apparatus and method for predicting intradialytic hypotension using a fitting curve of a pulse signal and information of an envelope thereof.

An intradialytic hypotension prediction apparatus according to an embodiment of the present disclosure comprises a processor; and a memory storing instructions executed by the processor, wherein the processor is configured to extract a preset parameter from a pulse signal representing a change in the pulse of a hemodialysis patient, and predict intradialytic hypotension by applying the parameter to a pre-trained prediction model.

The processor may extract feature information of the pulse signal using a fitting curve of the pulse signal and an envelope of the fitting curve, and extract the parameter based on the feature information.

The feature information may be extracted based on a difference between the pulse signal and the envelope, and a change in an interval slope of the pulse signal.

The feature information may include at least one of a first-order fitting curve obtained by performing a first-order fit on the pulse signal, a 19th-order fitting curve, an upper envelope connecting local maxima in the 19th-order fitting curve of the pulse signal, a lower envelope connecting local minima in the 19th-order fitting curve of the pulse signal, a difference between the upper envelope and the 19th-order fitting curve, a difference between the 19th-order fitting curve and the lower envelope, and a positive straight line or a negative straight line of a local pulse signal of the pulse signal.

The parameter may include at least one of statistical information of a pulse signal, an interval of difference between the upper envelope and the pulse signal, an interval of difference between the lower envelope and the pulse signal, a difference between an average pulse signal and E-points, a product of an average and a variance of Sub E-points, an overall slope of the pulse signal, and an area of a triangle formed by a slope of the local pulse signal, a horizontal line, and a vertical line.

The statistical information of the pulse signal may include at least one of an average and a variance of the pulse signal.

The interval of difference between the upper envelope and the pulse signal may include at least one of an interval where an area formed by a difference between the upper envelope and the pulse signal is largest, and an interval where the area formed by the difference between the upper envelope and the pulse signal is second largest.

The interval of difference between the lower envelope and the pulse signal may include at least one of an interval where an area formed by a difference between the pulse signal and the lower envelope is largest, and an interval where the area formed by the difference between the pulse signal and the lower envelope is second largest.

The area of the triangle formed by the slope of the local pulse signal, the horizontal line, and the vertical line may include at least one of the area of a triangle formed by largest negative slope of the local pulse signal, the area of a triangle formed by largest positive slope of the local pulse signal, the area of a triangle formed by second largest negative slope of the local pulse signal, the area of a triangle formed by second largest positive slope of the local pulse signal, an average area of triangles formed by negative slopes of the local pulse signal, and an average area of triangles formed by positive slopes of the local pulse signal.

An intradialytic hypotension prediction method according to another embodiment of the present disclosurecomprises: extracting, by a processor, a predetermined parameter from a pulse signal representing a change in the pulse of a hemodialysis patient; and predicting, by the processor, intradialytic hypotension by applying the parameter to a pre-trained prediction model.

In extracting the parameter, the processor may extract feature information of the pulse signal using a fitting curve of the pulse signal and an envelope of the fitting curve, and extracts the parameter based on the feature information.

The feature information may be extracted based on a difference between the pulse signal and the envelope, and a change in an interval slope of the pulse signal.

The feature information may include at least one of a first-order fitting curve obtained by performing a first-order fit on the pulse signal, a 19th-order fitting curve, an upper envelope connecting local maxima in the 19th-order fitting curve of the pulse signal, a lower envelope connecting local minima in the 19th-order fitting curve of the pulse signal, a difference between the upper envelope and the 19th-order fitting curve, a difference between the 19th-order fitting curve and the lower envelope, and a positive straight line or a negative straight line of a local pulse signal of the pulse signal.

The parameter may include at least one of statistical information of a pulse signal, an interval of difference between the upper envelope and the pulse signal, an interval of difference between the lower envelope and the pulse signal, a difference between an average pulse signal and E-points, a product of an average and a variance of Sub E-points, an overall slope of the pulse signal, and an area of a triangle formed by a slope of the local pulse signal, a horizontal line, and a vertical line.

The statistical information of the pulse signal may include at least one of an average and a variance of the pulse signal.

The interval of difference between the upper envelope and the pulse signal may include at least one of an interval where an area formed by a difference between the upper envelope and the pulse signal is largest, and an interval where the area formed by the difference between the upper envelope and the pulse signal is second largest.

The interval of difference between the lower envelope and the pulse signal may include at least one of an interval where an area formed by a difference between the pulse signal and the lower envelope is largest, and an interval where the area formed by the difference between the pulse signal and the lower envelope is second largest.

The area of the triangle formed by the slope of the local pulse signal, the horizontal line, and the vertical line may include at least one of the area of a triangle formed by largest negative slope of the local pulse signal, the area of a triangle formed by largest positive slope of the local pulse signal, the area of a triangle formed by second largest negative slope of the local pulse signal, the area of a triangle formed by second largest positive slope of the local pulse signal, an average area of triangles formed by negative slopes of the local pulse signal, and an average area of triangles formed by positive slopes of the local pulse signal.

The intradialytic hypotension prediction apparatus and method according to the above aspects of the present disclosure predicts intradialytic hypotension using a fitting curve of a pulse signal and information of an envelope thereof.

The intradialytic hypotension prediction apparatus and method according to the above aspects of the present disclosure predict intradialytic hypotension and notify a hemodialysis patient, a physician, or a guardian to enable treatment of the patient before hypotension occurs.

### BRIEF DESCRIPTION OF THE DRAWINGS

FIG. 1 is a block diagram of an intradialytic hypotension prediction apparatus according to the embodiment of the present disclosure.

FIG. 2A, FIG. 2B, FIG. 2C, and FIG. 2D are diagrams illustrating a pulse signal before the occurrence of intradialytic hypotension according to an embodiment of the present disclosure.

FIG. 3A, FIG. 3B, FIG. 3C, and FIG. 3D are diagrams illustrating parameters for predicting intradialytic hypotension according to an embodiment of the present disclosure.

FIG. 4 is a diagram illustrating parameter values for a patient with intradialytic hypotension according to an embodiment of the present disclosure.

FIG. 5 is a diagram illustrating parameter values for a normal patient according to an embodiment of the present disclosure.

FIG. 6 is a flowchart of a method for predicting intradialytic hypotension according to the another embodiment of the present disclosure.

### DETAILED DESCRIPTION

Hereinafter, embodiments of an intradialytic hypotension prediction apparatus and method according to an embodiment of the present disclosure will be described. In this process, the thickness of lines or the size of components illustrated in the drawings may be exaggerated for clarity and convenience of description. Furthermore, the terms described below are terms defined in consideration of functions in the present disclosure, and may vary depending on the intention or custom of a user or operator. Therefore, the definitions of these terms should be made based on the content throughout this specification.

The present disclosure may be implemented in various different forms and is not limited to the embodiments described herein. In the drawings, parts unrelated to the description are omitted to clearly describe the present disclosure, and similar reference numerals are assigned to similar parts throughout the specification.

Throughout the specification, when a part "includes" a certain component, it means that the part may further include other components, rather than excluding other components, unless there is a specific statement to the contrary.

The implementations described herein may be implemented, for example, as a method or process, an apparatus, a software program, a data stream, or a signal. Even if discussed only in the context of a single form of implementation (e.g., discussed only as a method), the implementation of the discussed features may also be implemented in other forms (e.g., an apparatus or a program). The apparatus may be implemented with appropriate hardware, software, firmware, and the like. The method may be implemented in an apparatus such as a processor, which generally refers to a processing device including, for example, a computer, a microprocessor, an integrated circuit, or a programmable logic device.

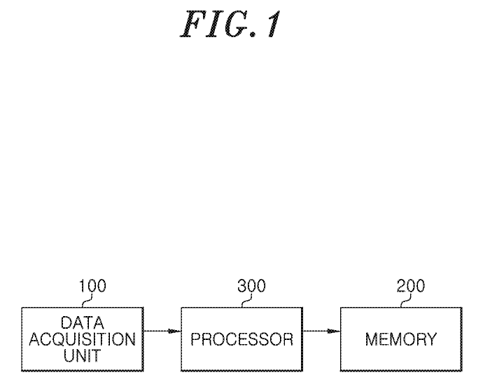

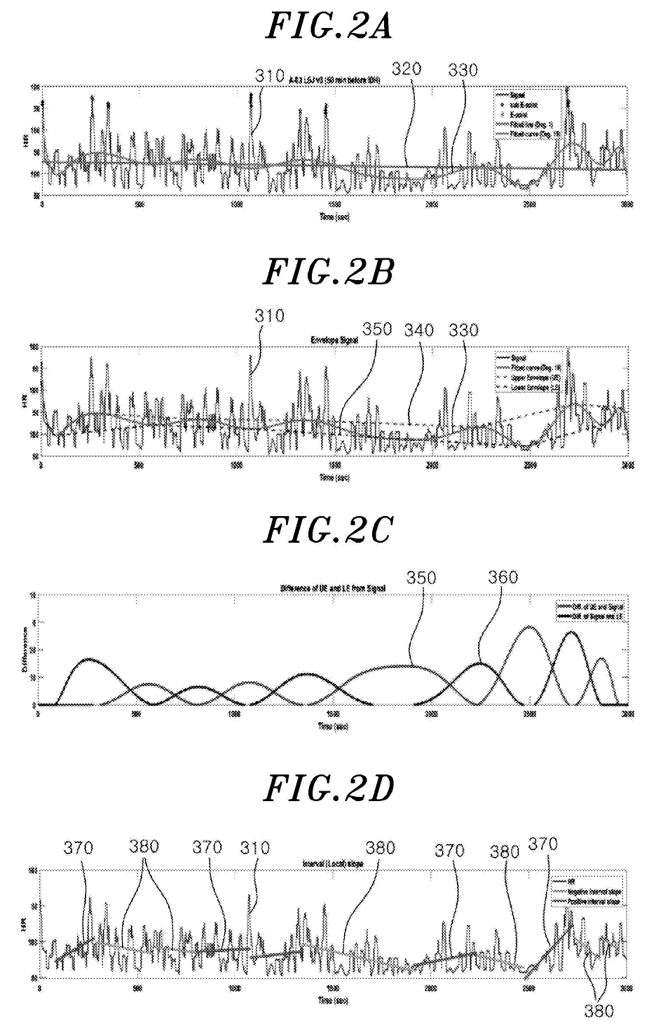

FIG. 1 is a block diagram of an intradialytic hypotension prediction apparatus according to the embodiment of the present disclosure, and FIG. 2 is a diagram illustrating a pulse signal before the occurrence of intradialytic hypotension according to an embodiment of the present disclosure.

Referring to FIGS. 1 and 2, an intradialytic hypotension prediction apparatus according to the embodiment of the present disclosure may include a data acquisition unit 100, a memory 200, and a processor 300.

The data acquisition unit 100 may acquire a pulse signal 310 of a hemodialysis patient. The data acquisition unit 100 may be a pulse sensor to measure the pulse signal 310 of the hemodialysis patient, a user interface device receiving the pulse signal 310 from a user, a database or storage device storing the pulse signal 310, or a communication interface device acquiring the pulse signal 310 through a communication network. Here, the communication network may be 3GPP (3rd Generation Partnership Project), LTE (Long Term Evolution), 5G (5th Generation), WIMAX (World Interoperability for Microwave Access), wired/wireless Internet, LAN (Local Area Network), Wireless LAN (Wireless Local Area Network), WAN (Wide Area Network), PAN (Personal Area Network), Bluetooth, Wi-Fi (Wireless Fidelity), and the like, but is not particularly limited thereto.

The memory 200 may store various data to be used by the processor 300. The data may include instructions for performing operations according to an embodiment of the present disclosure. That is, the memory 200 may store instructions for extracting a parameter for predicting intradialytic hypotension from the pulse signal 310 representing a change in the pulse of a hemodialysis patient and predicting intradialytic hypotension by applying the parameter to a prediction model. The parameter and the prediction model will be described later.

The memory 200 may include at least one storage medium among a Flash memory type, a Hard disk type, a Multimedia card micro type, a card type memory, Random Access Memory (RAM), Static Random Access Memory (SRAM), Read-Only Memory (ROM), Programmable Read-Only Memory (PROM), Erasable Programmable Read-Only Memory (EPROM), and Electrically Erasable Programmable Read-Only Memory (EEPROM).

The processor 300 may be coupled to the memory 200 and may execute instructions stored in the memory 200. The processor 300, by executing the instructions stored in the memory 200, may control at least one other component (e.g., a hardware or software component) coupled to the processor 300, and may perform various data processing or operations.

Furthermore, the processor 300 may be arranged such that the configuration for performing each function is separated at a hardware, software, or logic level. In this case, dedicated hardware for performing each function may be used. For this purpose, the processor 300 may be implemented as or include at least one of an Application Specific Integrated Circuit (ASIC), a Digital Signal Processor (DSP), Programmable Logic Devices (PLDs), Field Programmable Gate Arrays (FPGAs), a Central Processing unit (CPU), microcontrollers, and/or microprocessors.

The processor 300 may be implemented as a Central Processing Unit (CPU) or a System on Chip (SoC), and may drive an operating system or an application to control a plurality of hardware or software components coupled to the processor 300, and may perform various data processing and operations. The processor 300 may be configured to execute at least one instruction stored in the memory 200 and to store the execution result data in the memory 200.

The processor 300 may acquire the pulse signal 310 from the data acquisition unit 100.

The processor 300 may extract a predetermined parameter from the pulse signal 310 representing a change in the pulse, and may predict intradialytic hypotension by applying the parameter to a pre-trained prediction model.

Specifically, the processor 300 may obtain a fitting curve by fitting the pulse signal 310, and may extract feature information of the pulse signal 310 from the extracted fitting curve and an envelope.

The feature information of the pulse signal 310 may represent a difference between the pulse signal 310 of the hemodialysis patient and the envelope, and a change in an interval slope of the pulse signal 310. The feature information of the pulse signal 310 may be used to extract the parameter.

The feature information of the pulse signal 310 may include a first-order fitting curve 320 obtained by performing a first-order fit on the pulse signal 310, a 19th-order fitting curve 330 obtained by fitting the pulse signal 310 with a 19th-order term, an upper envelope (UE) 340 connecting local maxima in the 19th-order fitting curve 330 of the pulse signal 310, a lower envelope (LE) 350 connecting local minima in the 19th-order fitting curve of the pulse signal, a difference 350 between the upper envelope 340 and the 19th-order fitting curve 330, a difference 360 between the 19th-order fitting curve 330 and the lower envelope 350, and a positive straight line 370 or a negative straight line 380 of the local pulse signal 310 of the pulse signal 310.

FIG. 2(a) illustrates the pulse signal 310, a first-order fitting curve 320 of the pulse signal 310, that is, a fitted line (Degree 1), and a 19th-order fitting curve (fitted line (Degree 19)) 330.

FIG. 2(b) illustrates an upper envelope 340 connecting local maxima in the 19th-order fitting curve 330 of the pulse signal 310, and a lower envelope 350 connecting local minima in the 19th-order fitting curve 330 of the pulse signal 310.

FIG. 2(c) illustrates a difference 350 between the upper envelope 340 and the 19th-order fitting curve 330, and a difference 360 between the 19th-order fitting curve 330 and the lower envelope 350.

FIG. 2(d) illustrates a positive straight line 370 of the local pulse signal 310, and a negative straight line 380 of the local pulse signal 310.

As illustrated in FIG. 2, before the occurrence of intradialytic hypotension, a large difference occurs between the pulse signal 310 and each envelope due to a rapid change in the pulse, and it is understood that a large change in the interval slope of the pulse signal 310 is also observed.

The processor 300 may analyze the feature information of the pulse signal 310 to extract a parameter.

The parameter may include statistical information of a pulse signal, an interval representing the difference between the upper envelope 340 and the pulse signal, an interval representing the difference between the lower envelope 350 and the pulse signal, the difference between the average pulse signal and E-points (maximum points of momentary pulse compensation occurring to compensate for a decreased pulse), the product of the average and variance of Sub E-points (where Sub E-points are points included in the top one-third segment when the height from the signal average to an E-point is divided into three parts), the overall slope of the pulse signal (the slope of the first-order fitting curve 320), and the area of a triangle formed by the slope of the local pulse signal 310, a horizontal line, and a vertical line.

The statistical information of the pulse signal may include an average of the pulse signal and a variance of the pulse signal.

The interval representing the difference between the envelope and the pulse signal may include an interval where the area formed by the difference between the upper envelope 340 and the pulse signal is largest, and an interval where the area formed by the difference between the upper envelope 340 and the pulse signal is second largest.

The interval representing the difference between the lower envelope 350 and the pulse signal may be an interval where the area formed by the difference between the pulse signal and the lower envelope 350 is largest, and an interval where the area formed by the difference between the pulse signal and the lower envelope 350 is second largest.

The area of the triangle formed by the slope of the local pulse signal 310, the horizontal line, and the vertical line may include the area of a triangle formed by largest negative slope of the local pulse signal 310, a horizontal line, and a vertical line, the area of a triangle formed by largest positive slope of the local pulse signal 310, a horizontal line, and a vertical line, the area of a triangle formed by second largest negative slope of the local pulse signal 310, a horizontal line, and a vertical line, the area of a triangle formed by second largest positive slope of the local pulse signal 310, a horizontal line, and a vertical line, an average area of triangles formed by the negative slopes of the local pulse signal 310, a horizontal line, and a vertical line, and an average area of triangles formed by the positive slopes of the local pulse signal 310, a horizontal line, and a vertical line.

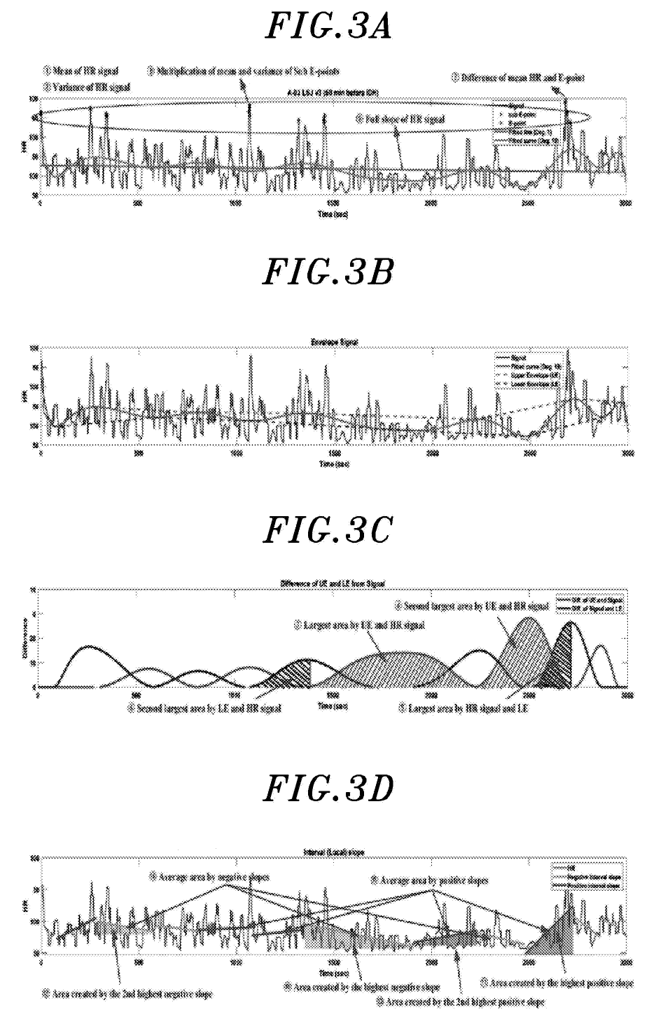

FIG. 3 is a diagram illustrating parameters for predicting intradialytic hypotension according to an embodiment of the present disclosure.

FIG. 3(a) illustrates an average of the pulse signal (①), a variance of the pulse signal (②), a difference between the average pulse signal and E-points (maximum points of momentary pulse compensation occurring to compensate for a decreased pulse) (⑦), a product of the average and variance of Sub E-points (⑧), and an overall slope of the pulse signal (⑨). Here, the Sub E-points may be points included in the top one-third segment when the height from the average of the pulse signal 310 to an E-point is divided into three parts to compensate for a decreased pulse.

FIG. 3(c) illustrates an interval where the area formed by the difference between the upper envelope 340 and the pulse signal is largest (③), an interval where the area formed by the difference between the upper envelope 340 and the pulse signal is second largest (④), an interval where the area formed by the difference between the pulse signal and the lower envelope 350 is largest (⑤), and an interval where the area formed by the difference between the pulse signal and the lower envelope 350 is second largest (⑥).

FIG. 3(d) may include the area of a triangle formed by largest negative slope of the local pulse signal 310, a horizontal line, and a vertical line (⑩), the area of a triangle formed by largest positive slope of the local pulse signal 310, a horizontal line, and a vertical line (⑪), the area of a triangle formed by second largest negative slope of the local pulse signal 310, a horizontal line, and a vertical line (⑫), the area of a triangle formed by second largest positive slope of the local pulse signal 310, a horizontal line, and a vertical line (⑬), an average area of triangles formed by the negative slopes of the local pulse signal 310, a horizontal line, and a vertical line (⑭), and an average area of triangles formed by the positive slopes of the local pulse signal 310, a horizontal line, and a vertical line (⑮).

The processor 300 may predict intradialytic hypotension by inputting the parameter to the prediction model.

The prediction model may be pre-trained to predict intradialytic hypotension by receiving the aforementioned parameter as input. The prediction model may be a Machine Learning model, an MLP (multilayer perceptron), or a CNN (Convolutional Neural Network), but is not particularly limited thereto.

Accordingly, the processor 300 may predict intradialytic hypotension by applying the parameter to the prediction model.

In this case, the processor 300 may detect a pulse conversion interval using the interval where the area formed by the difference between the upper envelope 340 and the pulse signal is largest (③), and the interval where the area formed by the difference between the upper envelope 340 and the pulse signal is second largest (④).

The processor 300 may detect a pulse change interval due to a compensation mechanism using the interval where the area formed by the difference between the pulse signal and the lower envelope 350 is largest (⑤), and the interval where the area formed by the difference between the pulse signal and the lower envelope 350 is second largest (⑥).

The processor 300 may calculate an average value of the pulse reduction interval due to intradialytic hypotension by using the average area of triangles formed by the negative slopes of the local pulse signal 310, a horizontal line, and a vertical line (⑭). The processor 300 may calculate an average value of the pulse increase interval due to a compensation mechanism by using the average area of triangles formed by the positive slopes of the local pulse signal 310, a horizontal line, and a vertical line (⑮).

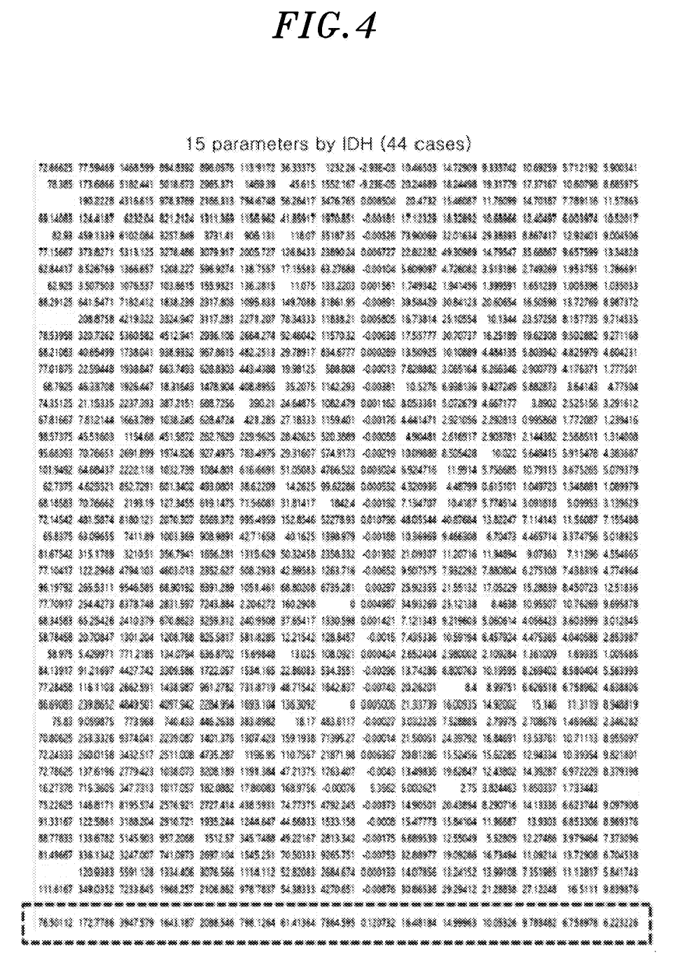

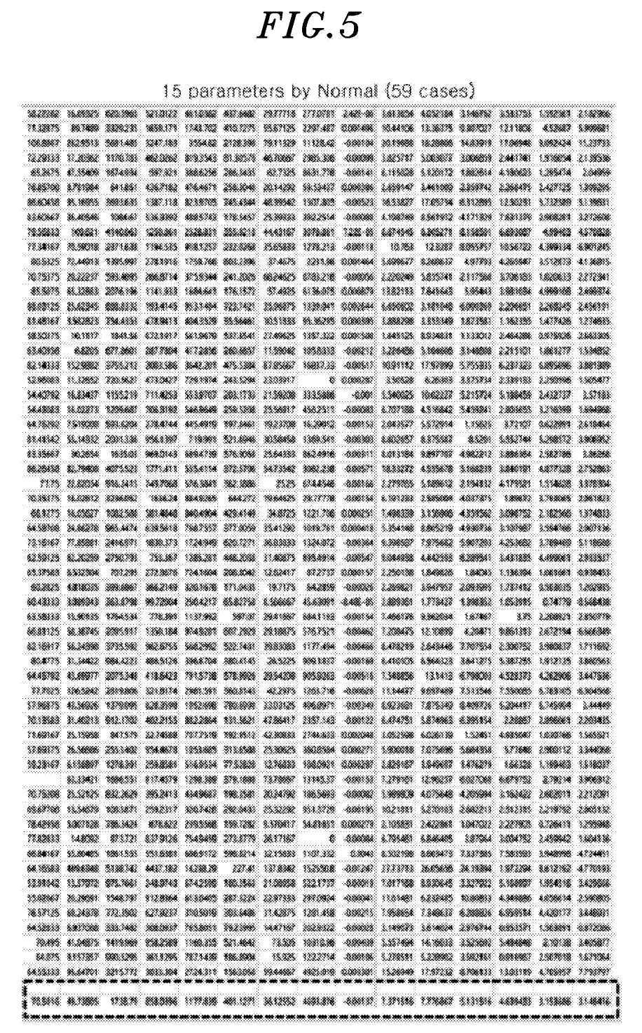

FIG. 4 is a diagram illustrating parameter values of a patient with intradialytic hypotension according to an embodiment of the present disclosure, and FIG. 5 is a diagram illustrating parameter values of a normal patient according to an embodiment of the present disclosure.

FIGS. 4 and 5 show the parameter values (average values) of a patient with intradialytic hypotension and the parameter values (average values) of a normal patient, and these 15 parameters may appear differently between a normal patient and a patient with intradialytic hypotension.

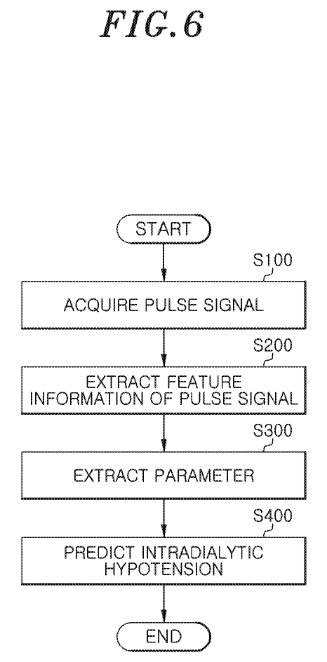

FIG. 6 is a flowchart of a method for predicting intradialytic hypotension according to the another embodiment of the present disclosure.

Referring to FIG. 6, first, the processor 300 may acquire the pulse signal 310 through the data acquisition unit 100 (S100). For example, the processor 300 may measure the pulse signal 310 of a hemodialysis patient through a pulse sensor, receive the pulse signal 310 through a user interface device, store the pulse signal 310 through a database or a storage device, or acquire the pulse signal 310 through a communication interface unit.

The processor 300 may obtain a fitting curve by fitting the pulse signal 310, and may extract feature information of the pulse signal 310 from the extracted fitting curve and an envelope (S200). The feature information of the pulse signal 310 may include a first-order fitting curve 320 obtained by performing a first-order fit on the pulse signal 310, a 19th-order fitting curve 330 obtained by fitting the pulse signal 310 with a 19th-order term, an upper envelope 340 connecting local maxima in the 19th-order fitting curve 330 of the pulse signal 310, a lower envelope 350 connecting local minima in the curve fitted to the pulse signal 310 with a 19th-order term, a difference 350 between the upper envelope 340 and the 19th-order fitting curve 330, a difference between the 19th-order fitting curve 330 and the lower envelope 350, and a positive straight line 370 or a negative straight line 380 of the local pulse signal 310 of the pulse signal 310.

The processor 300 may analyze the feature information of the pulse signal 310 to extract a parameter (S300). The parameter may include statistical information of a pulse signal, an interval of difference between the upper envelope 340 and the pulse signal, an interval of difference between the lower envelope 350 and the pulse signal, a difference between an average pulse signal and E-points, a product of an average and a variance of Sub E-points, an overall slope of the pulse signal, and an area of a triangle formed by the slope of the local pulse signal 310, a horizontal line, and a vertical line.

The processor 300 may predict intradialytic hypotension by inputting the parameter to a prediction model (S400). That is, the prediction model may predict intradialytic hypotension by receiving the aforementioned parameter as input.

As such, the intradialytic hypotension prediction apparatus and method according to an embodiment of the present disclosure may predict intradialytic hypotension using a fitting curve of the pulse signal 310 and information of an envelope thereof.

Furthermore, the intradialytic hypotension prediction apparatus and method according to an embodiment of the present disclosure predicts intradialytic hypotension and notifies a hemodialysis patient, a physician, or a guardian to enable treatment for the hemodialysis patient before hypotension occurs.

The present disclosure has been described with reference to the embodiments shown in the drawings, but these are merely exemplary, and it will be understood by those of ordinary skill in the art to which the technology pertains that various modifications and equivalent other embodiments are possible therefrom. Therefore, the technical scope of protection of the present disclosure should be determined by the patent claims below.

What is claimed is:

1. An intradialytic hypotension prediction apparatus comprising:
a processor; and
a memory storing instructions executed by the processor,
wherein the processor is configured to:
extract a predetermined parameter from a pulse signal representing a change in a pulse of a hemodialysis patient, and
predict intradialytic hypotension by applying the parameter to a pre-trained prediction model.

2. The intradialytic hypotension prediction apparatus of claim 1, wherein the processor extracts feature information of the pulse signal using a fitting curve of the pulse signal and an envelope of the fitting curve, and extracts the parameter based on the feature information.

3. The intradialytic hypotension prediction apparatus of claim 2, wherein the feature information is extracted based on a difference between the pulse signal and the envelope, and a change in an interval slope of the pulse signal.

4. The intradialytic hypotension prediction apparatus of claim 2, wherein the feature information includes at least one of a first-order fitting curve obtained by performing a first-order fit on the pulse signal, a 19th-order fitting curve, an upper envelope connecting local maxima in the 19th-order fitting curve of the pulse signal, a lower envelope connecting local minima in the 19th-order fitting curve of the pulse signal, a difference between the upper envelope and the 19th-order fitting curve, a difference between the 19th-order fitting curve and the lower envelope, and a positive straight line or a negative straight line of a local pulse signal derived from the pulse signal.

5. The intradialytic hypotension prediction apparatus of claim 4, wherein the parameter includes at least one of statistical information of the pulse signal, an interval of difference between the upper envelope and the pulse signal, an interval of difference between the lower envelope and the pulse signal, a difference between an average pulse rate and E-points, a product of an average and a variance of Sub E-points, an overall slope of the pulse signal, and an area of a triangle formed by a slope of the local pulse signal, a horizontal line, and a vertical line.

6. The intradialytic hypotension prediction apparatus of claim 5, wherein the statistical information of the pulse signal includes at least one of an average and a variance of the pulse signal.

7. The intradialytic hypotension prediction apparatus of claim 5, wherein the interval of difference between the upper envelope and the pulse signal includes at least one of an interval where an area formed by a difference between the upper envelope and the pulse signal is largest, and an interval where the area formed by the difference between the upper envelope and the pulse signal is second largest.

8. The intradialytic hypotension prediction apparatus of claim 5, wherein the interval of difference between the lower envelope and the pulse signal includes at least one of an interval where an area formed by a difference between the pulse signal and the lower envelope is largest, and an interval where the area formed by the difference between the pulse signal and the lower envelope is second largest.

9. The intradialytic hypotension prediction apparatus of claim 5, wherein the area of the triangle formed by the slope of the local pulse signal, the horizontal line, and the vertical line includes at least one selected from the group consisting of an area of a triangle formed by largest negative slope of the local pulse signal, an area of a triangle formed by largest positive slope of the local pulse signal, an area of a triangle formed by second largest negative slope of the local pulse signal, an area of a triangle formed by second largest positive slope of the local pulse signal, an average area of triangles formed by negative slopes of the local pulse signal, and an average area of triangles formed by positive slopes of the local pulse signal.

10. An intradialytic hypotension prediction method comprising:
extracting, by a processor, a predetermined parameter from a pulse signal representing a change in a pulse of a hemodialysis patient; and
predicting, by the processor, intradialytic hypotension by applying the parameter to a pre-trained prediction model.

11. The intradialytic hypotension prediction method of claim 10, wherein extracting the parameter includes:
extracting, by the processor, feature information of the pulse signal using a fitting curve of the pulse signal and an envelope of the fitting curve, and extracting the parameter based on the feature information.

12. The intradialytic hypotension prediction method of claim 11, wherein the feature information is extracted based on a difference between the pulse signal and the envelope, and a change in an interval slope of the pulse signal.

13. The intradialytic hypotension prediction method of claim 11, wherein the feature information includes at least one of a first-order fitting curve obtained by performing a first-order fit on the pulse signal, a 19th-order fitting curve, an upper envelope connecting local maxima in the 19th-order fitting curve of the pulse signal, a lower envelope connecting local minima in the 19th-order fitting curve of the pulse signal, a difference between the upper envelope and the 19th-order fitting curve, a difference between the 19th-order fitting curve and the lower envelope, and a positive straight line or a negative straight line of a local pulse signal derived from the pulse signal.

14. The intradialytic hypotension prediction method of claim 13, wherein the parameter includes at least one of statistical information of the pulse signal, an interval of difference between the upper envelope and the pulse signal, an interval of difference between the lower envelope and the pulse signal, a difference between an average pulse rate and E-points, a product of an average and a variance of Sub E-points, an overall slope of the pulse signal, and an area of a triangle formed by a slope of the local pulse signal, a horizontal line, and a vertical line.

15. The intradialytic hypotension prediction method of claim 14, wherein the statistical information of the pulse signal includes at least one of an average and a variance of the pulse signal.

16. The intradialytic hypotension prediction method of claim 14, wherein the interval of difference between the upper envelope and the pulse signal includes at least one of an interval where an area formed by a difference between the upper envelope and the pulse signal is largest, and an interval where the area formed by the difference between the upper envelope and the pulse signal is second largest.

17. The intradialytic hypotension prediction method of claim 14, wherein the interval of difference between the lower envelope and the pulse signal includes at least one of an interval where an area formed by a difference between the pulse signal and the lower envelope is largest, and an interval where the area formed by the difference between the pulse signal and the lower envelope is second largest.

18. The intradialytic hypotension prediction method of claim 14, wherein the area of the triangle formed by the slope of the local pulse signal, the horizontal line, and the vertical line includes at least one of the area of a triangle formed by largest negative slope of the local pulse signal, the area of a triangle formed by largest positive slope of the local pulse signal, the area of a triangle formed by second largest negative slope of the local pulse signal, the area of a triangle formed by second largest positive slope of the local pulse signal, an average area of triangles formed by negative slopes of the local pulse signal, and an average area of triangles formed by positive slopes of the local pulse signal.
